(19) United States
(12) Patent Application Publication
(10) Pub. No.: US 2025/0281375 A1
(43) Pub. Date: Sep. 11, 2025

(54) NON-AQUEOUS DENTIFRICE COMPOSITION

(71) Applicant: HALEON UK IP LIMITED, Weybridge, Surrey (GB)

(72) Inventors: Arwa MATOUG, Weybridge, Surrey (GB); Junior MENSUO, Weybridge, Surrey (GB); Nima ROOHPOUR, Weybridge, Surrey (GB); Corinne WEBSTER, Weybridge, Surrey (GB)

(21) Appl. No.: 18/710,253

(22) PCT Filed: Nov. 23, 2022
(86) PCT No.: PCT/EP2022/083049

§ 371 Date: May 15, 2024

(30) Foreign Application Priority Data
Nov. 29, 2021 (GB) 2117188.9

(51) Int. Cl. A61K 8/21; A61K 8/27; A61K 8/34; A61K 8/86; A61K 8/9717; A61Q 11/00

(52) U.S. Cl. CPC A61K 8/21; A61K 8/27; A61K 8/345; A61K 8/86; A61K 8/9717; A61Q 11/00

(57) ABSTRACT

A stable single phase non-aqueous dentifrice composition comprising stannous fluoride and at least one zinc salt. Preferably wherein the dentifrice composition comprises a toothpaste formulation.

### FIELD OF INVENTION

The present invention relates to a single phase non-aqueous dentifrice composition comprising at least one zinc salt and stannous fluoride. In particular the invention relates to a single phase non-aqueous dentifrice composition comprising zinc chloride and stannous fluoride.

### BACKGROUND TO THE INVENTION

The present invention is related to a stable, non-aqueous dentifrice comprising stannous fluoride and at least one zinc salt.

Stannous fluoride is well known for use in oral care applications. It has been proven to help protect against gingivitis, plaque formation and sensitivity.

Zinc is generally formulated into oral health products in order to reduce oral malodour, reduce plaque formulation and inhibit calculus formation.

The two actives have different chemical and physical properties which makes coformulation into a single phase of a dentifrice composition very challenging.

Combining these actives previously into a single toothpaste has usually required the use of different formulations being squeezed together on use. A striped toothpaste is commonly used for example. So that a different paste can be optimised for each active ingredient. And these are combined only briefly on the brush prior to use. This method allows for a single dentifrice containing the combined actives, but not without associated costs including the manufacture of separate formulations and the use of more complex tube, containment and nozzle arrangements.

It is the aim of this invention to provide a single-phase dentifrice composition that provides a stable storage until use environment for both stannous fluoride and zinc salts.

### SUMMARY OF INVENTION

In one aspect the invention provides a single phase non-aqueous dentifrice composition comprising at least one solvent, stannous fluoride, at least one zinc salt, a supporting polymer, at least one surfactant and carrageenan.

In a further aspect of the invention the at least one solvent comprises glycerol, polyethylene glycol (PEG) or a mixture thereof.

In a further aspect of the invention the at least one solvent comprises glycerol 30-70% by weight and polyethylene glycol (PEG) 10-30% by weight.

In a further aspect of the invention the at least one zinc salt comprises zinc chloride.

In a further aspect of the invention the at least one zinc salt comprises between 0.05% and 1% by weight of the composition.

In a further aspect of the invention the supporting polymer is a polyacrylic acid (PAA).

In a further aspect of the invention the PAA is a high molecular weight PAA polymer.

In a further aspect of the invention the supporting polymer comprises between 0.1 and 1.5% by weight of the composition.

In a further aspect of the invention the PEG has a molar mass between 380 and 420 grams per mol.

In a further aspect of the invention the carrageenan comprises between 0.1 and 1% by weight of the composition.

In a further aspect of the invention the at least one surfactant is selected from the group comprising sodium lauryl sulphate (SLS) and cocamidopropyl betaine.

In a further aspect of the invention the composition comprises both SLS and cocamidopropyl betaine.

In a further aspect of the invention the single phase nonaqueous dentifrice comprises
a) glycerol 52-55% by weight
b) PEG 400 18-22% by weight
c) stannous fluoride
d) zinc chloride 0.2-0.4% by weight
e) a high molecular weight PAA polymer 0.6-0.8% by weight
f) a 3:1 weight ratio of surfactants sodium laurel sulphate and cocamidopropyl betaine 1.0-2.4% by weight; and
g) carrageenan 0.1 to 0.4% by weight.

In a further aspect of the invention the composition further comprises an additional source of fluoride.

In a further aspect of the invention the composition comprises an anti-staining agent.

In a further aspect of the invention the composition comprises one or more additional ingredients selected from dental silicas, thickening silicas, flavourings, colourants, preservatives, pH modifiers, rheology modifiers, and abrasives.

The invention also comprises the use of the composition of any of the previous claims in combination with a toothbrush to clean teeth.

### DETAILED DESCRIPTION OF THE INVENTION

As used herein the term “dentifrice” includes any semi-solid preparation in the form of a paste, cream or gel for use in cleaning or treating all, or a portion of, the oral cavity of an individual.

As used herein the term “oral cavity” means an individual's teeth and gums including all periodontal regions including teeth down to the gingival margins and/or the periodontal pockets.

As used herein, the term “non-aqueous” means anhydrous or substantially free of water. The individual components of the non-aqueous composition may contain limited amounts of water as long as the overall composition remains substantially free of water. For the purposes of this disclosure the terms “non-aqueous” and “anhydrous” are interchangeable and have the same meaning.

As used herein the term “single phase” means the compositions are homogeneous and not a composition comprising two or more different compositions co-located.

The compositions of the present invention comprise stannous fluoride. Stannous fluoride is well known for use in oral care applications. It has been proven to help protect against gingivitis, plaque formation and sensitivity.

Stannous fluoride is highly water sensitive and in the presence of water in the mouth it is oxidised and breaks down to form stannic ions (Sn4+). These insoluble salts are what is believed to give the stannous fluoride its sensitivity benefits. The salts form a protective barrier over exposed dentine tubules.

To maximise the efficacy of stannous fluoride it is therefore necessary to keep it in an anhydrous environment prior to reaching the oral cavity.

Stannous fluoride levels in commercial dentifrices are highly controlled by regulation and differ in different parts of the world. The formulations of the present invention are stable to a wide range of different levels of stannous fluoride.

Suitably the composition comprises between 0.05% and 0.5% by weight stannous fluoride. Eg. 0.186% by weight (equating to 450ppm of fluoride), 0.382% by weight (equating to 927 ppm of fluoride), or 0.454% by weight (equating to 1150 ppm of fluoride).

The formulations of the invention may contain stannous fluoride up to 4% by weight.

The skilled person will be aware of the requirements for fluoride levels in different jurisdictions.

The non-aqueous dentifrice compositions of the present invention may also comprise additional sources of fluoride as required. Further non-limiting examples of additional fluoride include sodium fluoride, sodium monofluorophosphate, potassium fluoride, ammonium fluoride, bis-(hydroxethyl) amino-propyl-N-hydroxyethyloctadecylamine-dihydrofluoride and mixtures thereof.

Preferably any additional fluoride over the amount provided by stannous fluoride required in the formula will be provided by sodium fluoride, (NaF).

Zinc, in the form of ionic salts, is also well known for its use in oral health products. It is added to oral health compositions to reduce plaque formation and reduce malodour (halitosis) by chemically binding to volatile odour causing molecules and inhibiting calculus formation. Zinc also provides antibacterial properties.

Zinc salts are favourable for use in oral health products due to their low toxicity by oral ingestion.

The skilled person will be aware of the different forms of zinc that are used in dentifrice compositions. Non limiting examples of commonly used zinc salts in oral health products include, zinc chloride, zinc oxide, zinc citrate, zinc sulphate and zinc gluconate.

Being salts, these products are usually used in water based formulations. As it is very hard to achieve a non-aqueous formulation that is stable to charged ionic components like salts.

The non-aqueous dentifrice formulations of the present invention may comprise between 0.05 and 5% by weight of zinc salts, preferably 0.1 and 2.5% by weight and most preferably between 0.2 and 1.0% by weight of zinc salts.

The zinc salt in the present invention may comprise a single zinc salt or may comprise a mixture of two or more different zinc salts.

In a particularly preferred embodiment, the zinc salt comprises zinc chloride. And preferably the zinc chloride is present between 0.1% and 0.5% and more preferably between 02% and 0.4% by weight.

Zinc salts however also have their drawbacks. They are known for imparting an astringent taste at levels required for efficacy, and for interfering with surfactants and degrading the foaming and cleaning power of toothpastes. They also are very difficult to formulate in an anhydrous dentifrice. Their charged ionic character causes instability in non-aqueous formulations.

The applicants have surprisingly found an anhydrous dentifrice composition that is stable to both stannous fluoride and a zinc salt at effective levels for oral health benefits described above. The dentifrice also yields consumer acceptable organoleptic (taste and foaming) properties.

The applicants found during investigative work, that simply adding zinc salts to a number of different and known anhydrous dentifrice chassis, (known to be suitable for the stability of stannous fluoride) yielded unacceptable results. Even with very low levels of zinc salt, their addition to these compositions caused serious syneresis and/or rheological problems. Resulting in pastes with poor visual appeal, or poor viscosity, or completely unstable formulations causing the unacceptable loss of stannous over time.

The applicants were initially unable to solve these problems. Attempts were made simply by swapping between combinations of known gelling polymers and solvent mixtures. A number of different stabilizers, thickeners and additives were also trialled with no success.

The non-aqueous dentifrice compositions of the present invention may comprise any solvent non-aqueous/anhydrous solvents.

It was eventually surprisingly found that the addition of small amounts of carrageenan in combination with at least one solvent provided the required stable and effective anhydrous dentifrice, suitable for the delivery of zinc and stannous ions to the oral cavity.

In particular it was found that a solvent combination of glycerine and PEG with carrageenan provided the required stable and effective anhydrous dentifrice, suitable for the delivery of zinc and stannous ions to the oral cavity.

The non-aqueous dentifrice compositions of the present invention may comprise glycerine as the main solvent. The non-aqueous dentifrice compositions of the present invention may contain between 30-70% by weight of glycerine.

Glycerine is also known as glycerol and glycerin. The terms may be used interchangeably in this specification.

It is well known that commercially available glycerin may contain between 0.1-2.0% by weight of water which is in association with the glycerine. Typically, this amount is <0.5% and for example between 0.1-0.5% by weight of the glycerine. This small amount of water is bound to the glycerine and is therefore not available to the other ingredients. The skilled person would still consider a composition containing glycerine as being non-aqueous.

The solvent should in any case be as anhydrous as possible.

In a particularly preferred embodiment of the invention, the single phase non-aqueous dentifrice compositions comprise between 52-55% by weight of glycerine.

The solvent of the single phase non-aqueous dentifrice compositions of the present invention may also comprise polyethylene glycol (PEG).

The single phase non-aqueous dentifrices of the invention may comprise between 10-30% by weight of PEG.

PEG can be obtained in different chain lengths (FWs) with varying chemical and physical properties. Suitably for the compositions of the present invention, the polyethylene glycol may be selected from PEG 300, PEG 400 and mixtures thereof.

In one embodiment the polyethylene glycol comprises PEG 400. And in another preferred embodiment the PEG 400 comprises 18-22% of the weight of the non-aqueous dentifrice compositions of the present invention.

The at least one solvent of the single phase non-aqueous dentifrice of the present invention may comprise a mixture of glycerin and polyethylene glycol. Preferably the two solvents are present in a ratio, (Glycerine: PEG) of between, 1:1 and 4:1, by weight, more preferably 2:1 to 3:1, and most preferably between 2.3:1 to 2.7:1.

Other additional suitable solvents for use in the present invention include sorbitol, propylene glycol, polyethylene glycol or mixtures thereof.

The non-aqueous dentifrice composition according to the invention comprise a supporting (or gelling) polymer to solidify the solvent and actives.

The skilled person will be aware of many different types of supporting polymer for dentifrice use. Two non-limiting examples include a copolymer of a methyl vinyl ether and a maleic anhydride, such as Gantrez, and polyacrylic acid homopolymers, often referred to as Carbomers.

Suitably for the present invention the supporting (or gelling) polymer comprises a carboxyvinyl polymer such as a carbomer.

A carbomer comprises synthetic high molecular-weight cross-linked polymers of acrylic acid. The polymer chains formed of repeating units of acrylic acid may be cross-linked with, for example: allyl sucrose to provide a carbomer available commercially in one form as Carbopol™ 934; ethers of pentaerythritol to provide a carbomer available commercially in one form as Carbopol™ 974; or with divinyl glycol, available commercially in one form as Noveon™ AA-1. Carbopol™ polymers are manufactured by B.F. Goodrich Company.

In one embodiment the carboxyvinyl polymer comprises Carbopol™ 974.

The carboxyvinyl polymer may be present in the range of from about 0.1 to about 7.5% by weight of the non-aqueous composition. In one embodiment the carboxyvinyl polymer is present in an amount from about 0.3 to about 1.0% by weight of the composition.

The stable single phase non-aqueous dentifrice formulations of the present invention require the inclusion of carrageenan.

Carrageenan is an extract from a red seaweed commonly known as Irish Moss. This edible seaweed is native to the British Isles, where it's been used in traditional cooking for hundreds of years. It's also widely used in the food industry, mostly as a thickener and gelling agent.

Carrageenans contain 15-40% ester-sulphate content, which makes them anionic polysaccharides. They can be mainly categorized into three different classes based on their sulphate content. Kappa-carrageenan has one sulphate group per disaccharide, iota-carrageenan has two, and lambda-carrageenan has three.

The single phase non-aqueous dentifrice compositions of the invention can utilise any of the forms of carrageenan, either individually or as a mixture of two or more forms. Preferably, the non-aqueous single phase dentifrice compositions of the invention comprise iota-carrageenan, more preferably the carrageenan content of the non-aqueous single phase dentifrice compositions of the present invention comprises only the iota carrageenan form.

The single-phase anhydrous compositions of the present invention comprise between 0.05 and 2% by weight of carrageenan to allow for the formation of stable dentifrice compositions.

Preferably the carrageenan comprises between 0.1 and 1% by weight, more preferably between 0.2 and 0.5% by weight of the single phase non-aqueous dentifrice compositions.

Other gelling stabilizing agents may also be used in addition to carrageenan, but not in replacement of carrageenan. Other non-limiting examples of thickening gums that may use used include such as, but not limited to: xanthan gum, lecithin, guar gum, gellan gum, locust bean gum (carob bean gum) and Arabic gum (acacia gum).

The single phase non-aqueous dentifrice composition according to the invention comprises at least one surfactant. The at least one surfactant may comprise a single surfactant. Alternatively, the at least one surfactant may comprise a mixture of a first surfactant and a second surfactant. Alternatively, the at least one surfactant may comprise a mixture of a first surfactant, a second surfactant and a third and/or further surfactants.

A suitable surfactant for use in the compositions according to the invention belongs to the class of compounds known as betaines. Structurally, betaine compounds contain an anionic functional group such as a carboxylate functional group and a cationic functional group such as quaternary nitrogen functional group separated by a methylene moiety. They include n-alkyl betaines such as cetyl betaine and behenyl betaine, and n-alkylamido betaines such as cocamidopropyl betaine.

In one embodiment the betaine is cocamidopropyl betaine, commercially available under the trade name Tego Betain®.

Suitably the betaine is present in an amount ranging from about 0.05 to about 4% by weight of the single phase non-aqueous composition, for example from about 0.2 to about 2.0%, more preferably between about 0.3 to about 0.6% by weight of the non-aqueous composition.

Another surfactant for use in the single phase non-aqueous dentifrice compositions according to the invention is selected from a taurate based surfactant. Taurate surfactants useful in the present invention are salts of fatty acid amides of N-methyl taurine. They conform generally to the structural formula:

RC(O)N(CH₃)CH₂CH₂SO₃M

Where RC(O)— represents a fatty acid radical and M represents sodium,

potassium, ammonium or triethanolamine. Fatty acids having carbon chain lengths of from 10 to 20, including those derived from coconut, palm and tall oil are used. In one embodiment the fatty acid is derived from coconut. In one embodiment, sodium salts are used.

In one embodiment the taurate is sodium methyl cocyl taurate. This taurate surfactant is sold under the trademark by Adinol® CT by Croda.

The taurate surfactant may be present in an amount from about 0.1 to about 10% of the anhydrous dentifrice composition. In one embodiment the taurate surfactant is present in an amount from about 0.1 to about 5% by weight of the non-aqueous composition. In one embodiment the taurate surfactant is present in an amount from about 0.5 to about 2.0% by weight of the non-aqueous composition.

Another class of surfactants suitable for the present invention are alkyl sulphate surfactants of the following structural formula:

R¹OSO₃M

R1 represents a fatty alcohol moiety and M represents sodium, potassium, ammonium or triethanolamine. Fatty alcohols having carbon chain lengths of from about 10 to about 20, including those derived from coconut, palm oil and tall oil. In one embodiment, the fatty alcohol is lauryl alcohol. In one embodiment, a sodium salt is used.

In one embodiment the alkyl sulphate is sodium lauryl sulphate.

The alkyl sulphate surfactant may be present in an amount from about 0.1 to about 10% of the non-aqueous composition. In one embodiment the alkyl sulphate surfactant may be present in an amount from about 0.1 to about 5% by weight of the non-aqueous dentifrice composition.

In one embodiment the alkyl sulphate surfactant is present in an amount from about 0.5 to about 2.0% by weight of the non-aqueous composition.

In certain embodiments, the at least one surfactant comprises a first surfactant which is a C10-20 alkyl sulphate surfactant, and a second surfactant which consists of a betaine. Preferably wherein the first surfactant is sodium lauryl sulphate (SLS) and the second surfactant is cocamidopropyl betaine.

Preferably the ratio of SLS and cocamidopropyl betaine is between 2:1 and 4:1 by weight. Most preferably the ratio of the SLS to cocamidoproyl betaine is about 3:1.

In a particularly preferred embodiment, SLS comprises between 1.0 and 1.4% by weight and the cocamidopropyl betaine comprises between 0.3 and 0.5% by weight of the non-aqueous dentifrice composition.

An example single phase non-aqueous dentifrice of the present invention may comprise a composition as described in Table 1.

TABLE 1

| Ingredient | Function | % w/w |
|---|---|---|
| Glycerine | Formulation Solvent | 30-70% |
| Polyethylene Glycol | Formulation Solvent | 10-30% |
| At least one surfactant | Surfactant | 0.1-5% |
| Zinc salt(s) | active | >0.05% |
| Stannous Fluoride | active | >0.05% |
| Supporting polymer | Stabilizer | 0.2-2.0% |
| Carrageenan | Stabilizer | 0.1-1.0% |
| Optional ingredients | Multiple functions | 0.1-25% |

### Optional Ingredients

The single phase non-aqueous compositions of the invention may additionally (optionally) also contain one or more oral care active agents conventionally used in dentifrice formulations.

The skilled person will be aware of a great many additional ingredients that may be added to dentifrice formulations to provide or adjust beneficial properties.

Such agents may include, by way of non-limiting example: a desensitizing agent, an anti-calculus agent, an anti-erosion agent, a pH modifying ingredient, a rheology adjusting reagent, an antimicrobial agent, an anti-plaque agent, a whitening agent, an oral malodour agent or a mixtures thereof.

A dentally acceptable abrasive may optionally be added to the non-aqueous composition. Advantageously, the presence or absence of a dentally acceptable abrasive as well as the amount of such abrasive may be used to selectively control the abrasivity of the dentifrice composition made with the non-aqueous compositions of the invention. By further way of example, a desired amount of dentally acceptable abrasive may be added to increase the abrasivity of the overall non-aqueous composition.

Suitable abrasives for use in the non-aqueous composition include, for example, amorphous, gelled, precipitated or fumed silica, zinc orthophosphate, sodium bicarbonate (baking soda), plastic particles, alumina, hydrated alumina, calcium carbonate, calcium pyrophosphate, insoluble metaphosphates or mixtures thereof.

The silica abrasive may be a natural amorphous silica, for instance diatomaceous earth; or a synthetic amorphous silica such as a precipitated silica. By way of example, silica abrasives include those marketed under the following trade names Zeodent, Sident, Sorbosil or Tixosil by Huber, Degussa, Incos and Rhodia respectively.

Suitably a silica abrasive is present in an amount up to 25% by weight of the total composition, for example from 2 to 20% by weight for example from 5 to 15% by weight of the total composition.

Generally, an amount of abrasive suitable for use in the non-aqueous composition of the present invention will be empirically determined to provide an acceptable level of cleaning and polishing, in accordance with the techniques well known in the art. Suitably, the abrasive is present in an amount from about 0 to about 60%, typically from about 5 to about 30%, by weight of the non-aqueous composition.

Polyphosphates are known to help retard calculus formation and are examples of anti-calculus agents suitable for use in the invention. A polyphosphate is generally understood to consist of two or more phosphate groups arranged primarily in a linear configuration, although some cyclic derivatives may be present. Polyphosphates of use in the invention include pyrophosphates, polyphosphates having three or more polyphosphate groups such as sodium tripolyphosphate (STP), and polyphosphates having four or more polyphosphate groups such as tetrapolyphosphate and hexametaphosphate among others.

A desensitizing agent, including a tubule blocking agent or a nerve desensitizing agent and mixtures thereof, for example as described in WO 02/15809 (Block) may be included in a composition according to the invention. Such further optional desensitizing agent(s) include a strontium salt such as strontium chloride, strontium acetate or strontium nitrate or a potassium salt such as potassium citrate, potassium chloride, potassium bicarbonate, potassium gluconate and especially potassium nitrate. A desensitizing agent may also include a bioactive glass.

Compositions of the invention may further comprise an anti-crosion agent, for example a polymeric mineral surface-active agent as described in WO 04/054529 (Procter & Gamble).

Compositions of the present invention may contain additional formulating agents such as flavouring agents, sweetening agents, opacifying or colouring agents and preservatives, selected from those conventionally used in an oral hygiene composition art for such purposes.

In general, the optional agents may be used in a minor amount or proportion of the overall formulation. By way of example, such components are usually present in from about 0.001 to about 5% by weight of the non-aqueous composition. In total such components may range from 0.1 to 25% by weight of the composition.

The dentifrice composition typically has a viscosity suitable for application to the oral cavity. The viscosity will vary depending on the type of dentifrice composition made and the ultimate use thereof. One of skill in the art can readily prepare compositions with suitable viscosities for use in the oral cavity from the teachings provided herein.

Suitably a composition according to the invention may further comprise an inorganic thickening agent such as a thickening silica. Suitably, the thickening agent is a thickening silica, for example, a colloidal hydrated silica, available commercially for example as Sident 22S or Syloid 244FP.

In one embodiment the thickening silica is present in the range of from about 0 to about 15%, suitably from about 5.0 to about 15.0% by weight of the non-aqueous composition.

The compositions according to the present invention may be prepared by admixing the ingredients in the appropriate relative amounts in any order that is convenient.

A single phase non-aqueous dentifrice composition according to the present invention may be mildly acidic, neutral or mildly alkaline i.e., has a slurry pH in the range from 6.0 to 7.5, for example from pH 6.1 to 7.4, 6.2 to 7.3, or 6.2 to 7.2. In one embodiment the composition has a pH of about 6.2. In a further embodiment the composition has a pH of about 7.0. The pH referred to is that measured when the dentifrice composition is slurried with water in a 1:3 weight ratio of the composition to water. Suitably the slurry is prepared by slurring the dentifrice composition with water in a weight ratio of one part dentifrice composition and three parts distilled water. The pH is determined using a standard pH meter.

Table 2 describes a preferred general formulation of the non-aqueous dentifrice compositions of the invention.

TABLE 2

| Ingredient | Function | % w/w |
|---|---|---|
| Glycerine | Formulation Solvent | 52-55% |
| Polyethylene Glycol 400 | Formulation Solvent | 18-22% |
| SLS:Betaine (3:1) | Surfactants | 1-3% |
| Silicas | Thickener/Abrasive | 10-25% |
| STP | Anti-calculus agent | 3-8% |
| Stannous fluoride | Active | 0.1-1% |
| Zinc chloride | Active | 0.1-1% |
| Carbomer Homopolymer | Stabilizer | 0.4-1.5% |
| Carrageenan | Stabilizer | 0.1-0.5% |
| Optional ingredients | Multiple functions | 1-10% |

The non-aqueous dentifrice compositions of the present invention are stable over long periods. The compositions were all stable under conditions of 40° C. and 75% relative humidity for 3 months. By stable, this means that the formulation has no visible signs of breakdown or stress and that the active levels are maintained.

A specific example of a non-aqueous dentifrice composition of the present invention is revealed in table 3.

TABLE 3

| Ingredient | Function | % w/w |
|---|---|---|
| Glycerine | Formulation Solvent | 53.715% |
| PEG (Macrogol 400) | Formulation Solvent | 20.00% |
| Silicon Dioxide (Sident 225) | Thickener | 8.00% |
| Hydrated Silica (Zeodent | Thickener | 7.50% |
| 124) | | |
| STP | Anti-calculous agent | 5.0% |
| Sodium Lauryl Sulphate | Surfactant | 1.10% |
| (SLS) | | |
| Titanium dioxide | Opacifier | 1.0% |
| Carbomer Homopolymer | Stabilizer/polymer | 0.70% |
| (Carbopol 971) | | |
| Carrageenan | Stabiliser | 0.25% |
| NaF | Active | 0.0721% |
| Stannous Fluoride | Active | 0.454% |
| Cocamidopropyl betaine | Surfactant | 0.36% |
| Zinc chloride | Active | 0.3% |
| Colourant | Colourant | 0.003% |
| Sweetener | Sweetener | 0.35% |
| Flavour | Flavour | 1.2% |

1. A single phase non-aqueous dentifrice composition comprising:
a) at least one solvent;
b) stannous fluoride;
c) at least one zinc salt;
d) a supporting polymer;
e) at least one surfactant; and
f) carrageenan.

2. The single phase non-aqueous dentifrice composition according to claim 1, wherein the at least one solvent comprises glycerine, polyethylene glycol (PEG) or a mixture thereof.

3. The single phase non-aqueous dentifrice composition according to claim 1, wherein the at least one solvent comprises glycerine 30-70% by weight and polyethylene glycol (PEG) 10-30% by weight.

4. The single phase non-aqueous dentifrice composition according to claim 1, wherein the at least one zinc salt is zinc chloride.

5. The single phase non-aqueous dentifrice composition according to claim 1, wherein the at least one zinc salt comprises between 0.05% and 1% by weight of the composition.

6. The single phase non-aqueous dentifrice composition according to claim 1, wherein the supporting polymer is a polyacrylic acid (PAA).

7. The single phase non-aqueous dentifrice composition according to claim 6, wherein when the PAA is a carbopol homopolymer.

8. The single phase non-aqueous dentifrice composition according to claim 1, wherein supporting polymer comprises between 0.1 and 1.5% by weight of the composition.

9. The single phase non-aqueous dentifrice composition according to claim 3, wherein the PEG has a molar mass between 380 and 420 grams per mol.

10. The single phase non-aqueous dentifrice composition according to claim 1, wherein the carrageenan comprises between 0.1 and 1% by weight of the composition.

11. The single phase non-aqueous dentifrice composition according to claim 1, wherein the at least one surfactant is selected from the group comprising sodium lauryl sulphate (SLS) and cocamidopropyl betaine.

12. The single phase non-aqueous dentifrice composition according to claim 11, wherein the composition comprises both SLS and cocamidopropyl betaine.

13. The single phase non-aqueous dentifrice composition according to claim 1, wherein the dentifrice comprises:
a) glycerine, between 52-55% by weight;
b) PEG 400, between 18-22% by weight;
c) stannous fluoride;
d) zinc chloride, between 0.2-0.4% by weight;
e) a high molecular weight PAA polymer, between 0.6-0.8% by weight;
f) a 3:1 weight ratio of surfactants sodium laurel sulphate and cocamidopropyl betaine between 1.0-2.4% by weight; and
g) carrageenan, between 0.1 to 0.4% by weight.

14. The single phase non-aqueous dentifrice composition according to claim 1, wherein the composition further comprises an additional source of fluoride.

15. The single phase non-aqueous dentifrice composition according to claim 1, wherein the composition comprises an anti-staining agent.

16. The single phase non-aqueous dentifrice composition according to claim 1, wherein the composition comprises one or more additional ingredients selected from dental silicas, thickening silicas, flavourings, colourants, preservatives, pH modifiers, rheology modifiers, and abrasives.

17. The use of the single phase non-aqueous dentifrice composition of claim 1 in combination with a toothbrush to clean teeth.
